(19) United States
(12) Patent Application Publication
(10) Pub. No.: US 2016/0228560 A1
(43) Pub. Date: Aug. 11, 2016

(54) INJECTABLE PHARMACEUTICAL COMPOSITIONS FOR SEALING TISSUES

(71) Applicant: Thomas Q. Dinh, Minnetonka, MN (US)

(72) Inventor: Thomas Q. Dinh, Minnetonka, MN (US)

(21) Appl. No.: 15/017,670

(22) Filed: Feb. 7, 2016

Related U.S. Application Data
(60) Provisional application No. 62/113,408, filed on Feb. 7, 2015.

(51) Int. Cl. A61K 47/42; B65B 55/08; A61K 31/195; A61K 47/36; A61K 31/167; A61K 31/12; B65B 3/00; A61K 9/00

(52) U.S. Cl. CPC A61K 47/42; B65B 3/003; B65B 55/08; A61K 9/0019; A61K 47/36; A61K 31/167; A61K 31/12; A61K 31/195

(57) ABSTRACT

Various embodiments of a pharmaceutical composition for closing or sealing an opening or a bleeding site such as a vascular puncture site following percutaneous diagnostic or therapeutic interventional procedures or any other non-vascular conduits such fistulas formed within the organs of the body. The injectable composition includes a formulation comprised of a hemostatic compound and a bioadhesive that can be injected at a positioned adjacent to the puncture site in the vasculature, or injected into the non-vascular lumen to fill the conduit created at the puncture site. In some embodiments, the formulation also contains an analgesic agent to reduce pain at the puncture site and provide comfort to the patient. In other embodiments, the formulation contains a pro-healing agent to promote the healing process of the puncture site or the non-vascular conduit.

CROSS REFERENCE TO PENDING APPLICATIONS

This application claims the benefit of U.S. Provisional Application No. 62/113,408, filed Feb. 7, 2015.

### FIELD OF THE INVENTION

The present invention relates generally to injectable pharmaceutical compositions and their method of manufacture, and more specifically, the disclosure relates to compounds with a combination of pharmaceutical agents to simultaneously treat multiple effects of procedures and the treatment of various disorders.

### BACKGROUND

In vascular applications, minimally invasive interventional procedures such as balloon angioplasty and stenting are commonly employed to treat patients with vascular obstructions via the vasculature of the human body. To gain access to the vasculature, the Seldinger technique is commonly employed to access the desired blood vessel, usually the femoral artery or the radial artery (in case of brachial approach). This involves using a small gauge hollow needle to insert through the skin and access the desired blood vessel. A guidewire is then inserted through the needle into the vessel and the needle is removed. Next, a dilator and an introducer sheath are advanced together over the guidewire through the skin and into the vessel.

After confirming that the access to the vessel has been established, the dilator and guidewire are then removed leaving the introducer sheath in place to be used as a conduit for subsequent procedures. The introducer sheath usually has a rubber valve at the proximal end to prevent blood loss of the patient during the procedure.

Following the procedure, the introducer sheath is removed from the artery and manual pressure is applied directly to the skin. The pressure is focused above the access puncture for about thirty minutes to inhibit blood loss until the body's natural clotting process seals the puncture. This technique is generally cumbersome because it is uncomfortable for the patient and requires a significant amount of operating room time.

Many sealing technologies and devices have been developed to stop the bleeding more quickly with a secondary goal of reducing operating time. These technologies and devices range from mechanical suturing devices to collagen plugs, vascular clips, staples, and the use of adhesives and sealants. Most showed improvement over the manual compression method, but with varying degrees of success and ease of use.

Most recently, vascular closure devices have been developed that deposit a plug outside the vessel (extra-arterial) without the entry of a component inside the vessel. Such devices generally require a consistent placing of the plug near the arterial wall. These devices suffer from a number of drawbacks. For example, if the plug did not solidify quickly and bond to the tissue surrounding the puncture site, it will not prevent leakage. In addition, since the plug did not form a strong bond with the tissue, the pressure exerted on the plug can cause the plug to move away from the hole in the vessel resulting in a hematoma or other complication at the puncture site.

As a result, it is desirable to provide an improved sealing formulation that is easy to use, seals quickly and forms a strong bond with tissues without leaving a component in the blood vessel.

On the non-vascular side, it is sometimes required to seal a non-vascular conduit that forms a canal or a small tract from an organ to outside of the patient's body, or a canal in between organs. The transport of undesired biological materials to outside of the body or in between the organ may cause some detrimental effects to the organ and also a reduction in the quality of life for some patients. For example, anal fistula, or fistula-in-ano, is a chronic inflammation of the perianal area that can cause significant decrease in the quality of life for patients. The primary goal in managing anal fistula is to eliminate the fistula tract by closing the internal and external opening while preserving anal sphincter function. Treatment of anal fistula is still mainly surgical including fistulotomy, cutting setons, staged fistulotomy, and endoanal advancement flaps. However, the major limitations of these surgical procedures include a significant risk of incontinence, with minor incontinence ranging from 36% to 63% for cutting seton, and up to 31% for advancement flaps. They may also leave the patient with an open wound that can take weeks or months to heal. Collagen plugs, developed by Bard and Gore Medical, have been used to treat anal fistula, but these devices face high incidences of plug dislodgement from the fistula site. An injectable formulation with an ability to form a plug within the fistula track that may also conform to the contour of the track while simultaneously bonding strongly to the surface tissue would be a desirable treatment and improvement of currently practiced methods.

Perianal fistulas also are a common complication of Crohn's disease affecting 14-38% patients. Currently there are more than 500,000 Crohn's disease patients in the US with an incidence of between 3.1 to 14.6 cases per 100,000 person—years (Loftus et al., Alimentary Pharmacology & Therapeutics, 2002). For Crohn's disease patients, despite improvements in medical therapy, fistulas rarely heal and frequently require surgical intervention. Attempts to fill fistula tracts with fibrin glue sealants have primarily been used to treat complicated fistulas in which simple fistulotomy is deemed to carry a significant risk of incontinence. Despite a lack of efficacy with fibrin sealants, the use of a filler material remains an attractive goal for the treatment of patients with complex anal fistulas. Potential advantages of this technique include simple and repeatable application, preservation of sphincter function, minimal patient discomfort, and the ability for subsequent surgical options, if needed.

### BRIEF SUMMARY OF THE INVENTION

This invention discloses an injectable formulation in the form of viscous paste for stopping a bleeding at a vascular puncture site such as a blood vessel or sealing a non-vascular conduit that connects between two organs or two anatomical regions in the body. The formulation disclosed herein is also designed to improve upon a fibrin sealant and serve as the next option for the treatment of such fistulas.

### BRIEF DESCRIPTION OF THE DRAWINGS

FIG. 1 shows a collagen paste formulation of a pharmaceutical composition after all components were mixed.

FIG. 2 shows the formulation after it is being loaded into a delivery system such as a syringe.

FIG. 3 shows a form of a plug after the formulation is injected from the delivery system.

### DETAILED DESCRIPTION OF THE INVENTION

According to its major aspects and broadly stated, some embodiments of the present invention provide methods and compositions for injectable sealing formulations. The sealing formulations are bioadhesive pastes with hemostatic properties intended to stop bleeding by closing a puncture site on a large blood vessel following percutaneous diagnostic or interventional cardiology procedures. The injectable pastes may be used to seal other non-vascular tracts that form between the organs or between the organ and outside of the body.

In one embodiment of the invention, porcine dermis is decellularized by processing the dermis through a chemical process that removes all cells from the porcine dermis. The decellularized dermis is less likely to cause an immune reaction if implanted in an area of human body.

In another embodiment of the invention, the decellularized porcine dermis is homogenized and filtered through a sieve to collect collagen particles with sizes of less than or equal to 150 microns. The collagen particles are then treated with a solution of water-ethanol or water-isopropanol to partially sterilize the collagen particles. The solution is then filtered and the collagen particles are re-suspended in sterile water and frozen. The frozen mixture of collagen in water is lyophilized to obtain a dry powder form of collagen particles.

In some embodiments of the invention, the injectable sealing composition has a formulation comprised of a paste of lyophilized mixture of collagen powder or fiber, a bioadhesive and a pharmaceutical agent in sterile saline and lactic acid. The bioadhesive is chitosan with a molecular weight (MW) of greater than 500 Kilo Daltons.

In some embodiments of the invention, the injectable sealing composition has formulation comprised of a paste of lyophilized mixture of collagen powder or fiber, a bioadhesive and a pharmaceutical agent in sterile saline and lactic acid. The bioadhesive is chitosan with a molecular weight (MW) of greater than 500 Kilo Daltons and the pharmaceutical agent is lyophilized thrombin or transxenamic acid.

In some embodiments of the invention, the injectable sealing composition has a formulation comprised of a paste of lyophilized mixture of collagen powder or fiber, a bioadhesive and a pharmaceutical agent in sterile saline and lactic acid. The bioadhesive is chitosan with a molecular weight (MW) of greater than 500 Kilo Daltons and the pharmaceutical agent is lidocaine.

In some embodiments of the invention, the injectable sealing composition has a formulation comprised of a paste of lyophilized mixture of collagen powder or fiber, a bioadhesive and a pharmaceutical agent in sterile saline and lactic acid. The bioadhesive is chitosan with a molecular weight (MW) of greater than 500 Kilo Daltons and the pharmaceutical agent is curcumin and its derivatives.

In some embodiments of the invention, the injectable sealing composition has a formulation comprised of a paste of lyophilized mixture of collagen powder or fiber, a bioadhesive and a pharmaceutical agent in sterile saline and lactic acid. The bioadhesive is chitosan with a molecular weight (MW) of greater than 500 Kilo Daltons and the pharmaceutical agent is an anti-inflammatory agent. The anti-inflammatory agent can be selected from the following pharmaceuticals including: sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, β-estradiol, rosiglitazone, troglitazone, pioglitazone, S-nitrosoglutathione, gliotoxin G, panepoxydone, cycloepoxydon tepoxalin, or a combination thereafter.

In some embodiments of the invention, the injectable sealing composition has a formulation comprised of a paste of lyophilized mixture of collagen powder or fiber, a bioadhesive and a pharmaceutical agent in sterile saline and lactic acid. The bioadhesive is chitosan with a molecular weight (MW) of greater than 500 Kilo Daltons and the ratio of collagen to chitosan is 20 to 1 by weight and preferably 10 to 1 by weight.

In some embodiments of the invention, the injectable sealing composition has a formulation comprised of a paste of lyophilized mixture of collagen powder or fiber, a bioadhesive and a pharmaceutical agent in sterile saline and lactic acid. The bioadhesive is chitosan with a molecular weight (MW) of greater than 500 Kilo Daltons and the pharmaceutical agent is lyophilized thrombin and the concentration of thrombin is between 0.1 units/ml to 30 units/ml of the formulation mixture and preferably 0.1 to 5 units/ml of formulation mixture.

In some embodiments of the invention, the injectable sealing composition has a formulation comprised of a paste of lyophilized mixture of collagen powder or fiber, a bioadhesive and a pharmaceutical agent in sterile saline and lactic acid. The bioadhesive is chitosan with a molecular weight (MW) of greater than 500 Kilo Daltons and the pharmaceutical agent is lyophilized transxenamic acid and the concentration of transxenamic acid is between 0.01 mg/ml to 50 mg/ml of the formulation mixture.

In some embodiments of the invention, the injectable sealing composition has a formulation comprised of a paste of lyophilized mixture of collagen powder or fiber, a bioadhesive and a pharmaceutical agent in a solution comprised of sterile saline and lactic acid. The bioadhesive is chitosan with a molecular weight (MW) of greater than 500 Kilo Daltons and the pharmaceutical agent is lidocaine and the concentration of lidocaine is between 0.1 mg/ml to 50 mg/ml of formulation mixture.

In some embodiments of the invention, the injectable sealing composition has a formulation comprised of a paste of lyophilized mixture of collagen powder or fiber, a bioadhesive and a pharmaceutical agent in sterile saline and lactic acid. The bioadhesive is chitosan with a molecular weight (MW) of greater than 500 Kilo Daltons and the pharmaceutical agent is curcumin and the concentration of curcumin is between 0.1 mg/ml to 50 mg/ml of formulation mixture.

In some embodiments of the invention, the concentration of the anti-inflammatory agent varies from 0.1 mg/ml of mixture to 50 mg/ml of mixture. The anti-inflammatory agent includes sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, β-estradiol, rosiglitazone, troglitazone, pioglitazone, S-nitrosoglutathione, gliotoxin G, panepoxydone, cycloepoxydon tepoxalin, or a combination thereafter.

In some embodiments of the invention, the injectable sealing composition/formulation is comprised of a paste of lyophilized mixture of collagen powder or fiber, a bioadhesive, and a pharmaceutical agent in a solution of sterile saline and lactic acid. The concentration of lactic acid in sterile saline is 0.3 to 3% by volume.

This invention discloses a method of preparing a lyophilized porcine collagen powder or microfiber, the method comprising: obtaining decellulized porcine dermis, grinding the porcine dermis into paste, suspending the paste in a liquid carrier of sterile water or a mixture of sterile water and alcohol, and filtering the suspension through a 150 micron sieve to obtain collagen particles of sizes less than 150 micron, suspending the particles in sterile water, freezing the particle suspension and lyophilizing the frozen suspension to obtain the collagen powder or microfiber.

In some embodiments of this invention, the invention discloses a method of preparing an injectable sealing composition/formulation, the method comprising: mixing lyophilized collagen powder or microfiber as described in this invention with an aqueous solution of chitosan and lactic acid in sterile saline to form a paste, adding a pharmaceutical agent to improve the sealing function or providing comfort to the patient, loading the final paste mixture into individual syringe, packaging the final syringe in a sterilization pouch and then sterilizing the final product.

In some embodiments of this invention, the invention discloses a method of preparing an injectable sealing composition/formulation, the method comprising: mixing lyophilized collagen powder or microfiber as described in this invention with an aqueous solution of chitosan and lactic acid in sterile saline to form a paste, adding a pharmaceutical agent such as thrombin or transxenamic acid to improve the sealing function, loading the final paste mixture into individual syringes, packaging the syringes in a sterilization pouch and then sterilizing the final product.

In some embodiments of this invention, the invention discloses a method of preparing an injectable sealing composition/formulation, the method comprising: mixing lyophilized collagen powder or microfiber as described in this invention with an aqueous solution of chitosan and lactic acid in sterile saline to form a paste, adding a pharmaceutical agent such as lidocaine to provide comfort to the patient, loading the final paste mixture into individual syringes, packaging the syringes in a sterilization pouch and then sterilizing the final product.

In some embodiments of this invention, the invention discloses a method of preparing an injectable sealing composition/formulation, the method comprising: mixing lyophilized collagen powder or microfiber as described in this invention with an aqueous solution of chitosan and lactic acid in sterile saline to form a paste, adding a pharmaceutical agent such as curcumin and/or its derivatives to provide a mean for better healing, loading the final paste mixture into individual syringes, packaging the syringes in a sterilization pouch and then sterilizing the final product.

In some embodiments of this invention, the invention discloses a method of preparing an injectable sealing composition/formulation, the method comprising: mixing lyophilized collagen powder or microfiber with an aqueous solution of chitosan and lactic acid in sterile saline to form a paste, adding an anti-inflammatory agent wherein the anti-inflammatory agent is selected from the group of sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, β-estradiol, rosiglitazone, troglitazone, pioglitazone, S-nitrosoglutathione, gliotoxin G, panepoxydone, cycloepoxydon tepoxalin or a combination thereafter, loading the final paste mixture into individual syringes, packaging the individual syringes in a sterilization pouch, and then sterilizing the final product.

In some embodiments of the invention, the invention discloses a method of preparing an injectable sealing composition/formulation, the method comprising: mixing lyophilized collagen powder or microfiber with an aqueous solution of chitosan and lactic acid in sterile saline to form a paste, adding a pharmaceutical agent such as curcumin and/or its derivatives to provide comfort to the patient, loading the final paste mixture into individual syringes, packaging the individual syringes in a sterilization pouch, and then sterilizing the final product wherein the method of sterilization is Electronic beam (E-beam).

In some embodiments of this invention, the invention discloses a method of preparing an injectable sealing composition/formulation, the method comprising: mixing lyophilized collagen powder or microfiber with an aqueous solution of chitosan and lactic acid in sterile saline to form a paste, adding an anti-inflammatory agent wherein the anti-inflammatory agent is selected from a group of wherein the anti-inflammatory agent is selected from the group of sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, β-estradiol, rosiglitazone, troglitazone, pioglitazone, S-nitrosoglutathione, gliotoxin G, panepoxydone, cycloepoxydon tepoxalin or a combination thereafter, loading the final paste mixture into individual syringes, packaging the individual syringes in a sterilization pouch, and then sterilizing the final product wherein the method of sterilization is Electronic beam (E-beam).

In some embodiments of this invention the chitosan in the mixture is crosslinked by E-beam radiation to increase viscosity of the mixture during the E-beam sterilization process.

According to one embodiment, a method of sealing a puncture in a blood vessel comprises: injecting the composition/formulation to quickly form a plug outside of the blood vessel and adjacent to the puncture site, the plug being reacted to the surrounding tissues to form a strong bond with the tissues and to prevent the plug from moving away from the blood vessel. The plug may contain a bioadhesive and a pharmaceutical agent that attracts and reacts the red blood cells and other clotting proteins such as fibrinogen and factor XIII.

According to one embodiment, a method of sealing a conduit or a fistula comprises injecting the composition/formulation to form a plug inside of the conduit or the fistula tract, the plug being reacted to the surrounding tissues to form a strong bond with the tissues and to prevent the plug from dislodging from the conduit or the tract. The plug may contain a bioadhesive to improve tissue adhesion and a pharmaceutical agent that can treat the inflammation condition of the tract.

According to another embodiment, the formulation or pharmaceutical composition described herein is biocompatible and bioabsorbable.

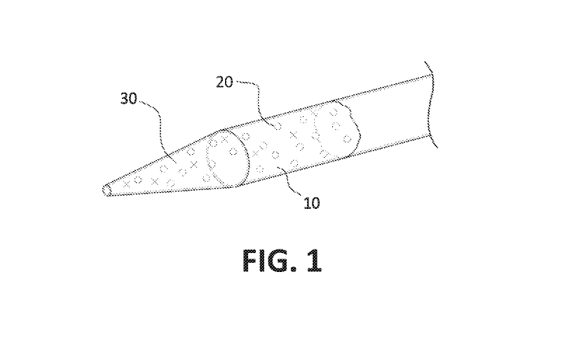

The sealing composition/formulation described in this invention can be used to seal any vascular bleeding or any nonvascular conduit that can transport any undesirable materials between organs or tissues. As shown in FIG. 1, the sealing composition/formulation is in the form of injectable paste. The injectable paste 10 consists of collagen particles 20 of sizes of 150 microns and smaller and the bioadhesive chitosan 30. The bioadhesive chitosan is dissolved in a solution of sterile water or 0.9% saline containing a small amount of lactic acid prior to mixing with the collagen particles to form an injectable paste. The total weight percent of collagen particles is usually between 0.5 to 2 percent and preferably between 0.5 to 10 percent for more viscous formulations. The composition/formulation can be manufactured aseptically or can be packaged for sterilization.

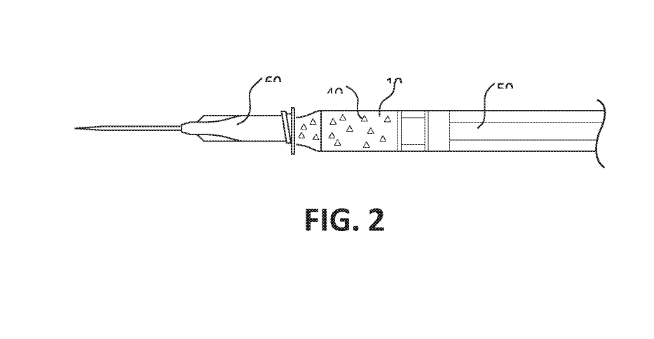

FIG. 2 shows the injectable composition/formulation of the sealing product and a form of a delivery system. The sealing composition/formulation 10 is loaded in a syringe 50 with a cannula or a needle 60 for access to area or tissue for sealing. FIG. 2 also shows that the composition/formulation contains a pharmaceutical compound 40 for anti-inflammation and healing. In this example, the pharmaceutical agent is curcumin, which is an anti-inflammatory drug. The content of anti-inflammatory drug can vary from 20 micrograms to 2000 microgram per cubic centimeter (or ml) of paste composition/formulation. The delivery system and the formulation can be packaged and sterilized by a radiation sterilization method like E-beam or gamma radiation sterilization.

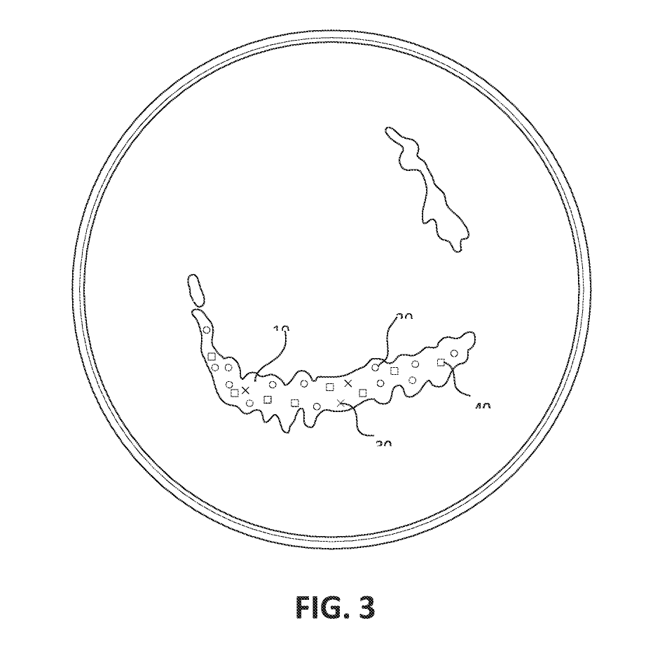

FIG. 3 shows the injectable composition/formulation of the sealing product described in this invention after it was delivered from the delivery system. The paste composition/formulation 10 that contains collagen particles 20, bioadhesive chitosan 30 and a pharmaceutical compound 40 can be conformed to any shape and contour to accommodate any anatomical configuration. The pharmaceutical compound in this example is curcumin. However, it is advisable that any pharmaceutical agent including anti-inflammatory agents, clotting agents, mtor inhibitors, NF-kβ inhibitors, proteins, growth factors, antibodies, genes, DNA, RNA, and stem cells can be used either as a single active agent or in combination of two or more pharmaceutical ingredients.

What is claimed is:

1. An injectable sealing composition comprised of:
a formulation containing a paste of lyophilized mixture of collagen powder or fiber, a bioadhesive and a pharmaceutical agent.
The injectable sealing composition is used for sealing vascular puncture sites and other non-vascular hollow conduits.

2. The injectable composition of claim 1, wherein the collagen powder is processed from decellularized porcine dermis.

3. The injectable composition of claim 1, wherein the bioadhesive is chitosan with a molecular weight (MW) of greater than 500 Kilo Daltons.

4. The injectable composition of claim 1, wherein the pharmaceutical agent is lyophilized thrombin or transxenamic acid.

5. The injectable composition of claim 1, wherein the pharmaceutical agent is lidocaine.

6. The injectable composition of claim 1, wherein the pharmaceutical agent is curcumin and its derivatives

7. The injectable composition of claim 1, wherein the pharmaceutical agent is an anti-inflammatory agent.

8. The injectable composition of claim 1, wherein the solvent for the mixture is sterile saline and lactic acid.

9. The injectable composition of claim 1, wherein the ratio of collagen to chitosan is 20 to 1 by weight and preferably 5 to 1 by weight.

10. The injectable composition of claim 4, wherein the concentration of thrombin is between 0.1 units/ml to 30 units/ml of the formulation mixture and preferably 0.1 to 5 units/ml of formulation mixture.

11. The injectable composition of claim 4, wherein the concentration of transxenamic acid is between 0.01 mg/ml to 50 mg/ml of the formulation mixture.

12. The injectable composition of claim 5, wherein the concentration of lidocaine is between 0.1 mg/ml to 50 mg/ml of hemostatic mixture.

13. The injectable composition of claim 6, wherein the concentration of curcumin is between 0.1 mg/ml to 50 mg/ml of hemostatic mixture.

14. The injectable composition of claim 7, wherein the anti-inflammatory agent is sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, β-estradiol, rosiglitazone, troglitazone, pioglitazone, S-nitrosoglutathione, gliotoxin G, panepoxydone, cycloepoxydon tepoxalin, or a combination thereafter.

15. The injectable composition of claim 8, wherein the concentration of lactic acid in sterile saline is 0.3 to 3% by volume.

16. A method of preparing a lyophilized porcine collagen powder or microfiber, the method comprising:
obtaining decellularized porcine dermis;
grinding the porcine dermis into paste;
suspending the paste in sterile water;
filtering the suspension through a 150 micron sieve to obtain collagen particles of sizes less than 150 micron;
freezing the particle suspension;
and lyophilizing the frozen suspension to obtain the collagen powder or microfiber.

17. A method of preparing an injectable sealing composition, the method comprising:
mixing decellularized porcine and lyophilized collagen powder or microfiber with an aqueous solution of chitosan and lactic acid in sterile saline to form a paste;
adding a pharmaceutical agent to improve the sealing function or providing comfort to the patient;
loading the final paste mixture into individual syringes, packaging the individual syringes in a sterilization pouch;
and then sterilizing the final product.

18. The method as in claim 17, wherein the pharmaceutical agent is thrombin or transxenamic acid

19. The method as in claim 17, wherein the pharmaceutical agent is lidocaine.

20. The method as in claim 17, wherein the pharmaceutical agent is curcumin and its derivatives.

21. The method as in claim 17, wherein the pharmaceutical agent is an anti-inflammatory agent.

22. The method as in claim 21, wherein the anti-inflammatory agent is sulfasalazine, sulindac, indomethacin, diclofenal, etodolac, meclofenate, mefenamic acid, nambunetone, piroxicam, phenylbutazone, meloxicam, dexamethasone, betamethasone dipropionate, diflorsasone diacetate, clobetasol propionate, halobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, β-estradiol, rosiglitazone, troglitazone, pioglitazone, S-nitrosoglutathione, gliotoxin G, panepoxydone, cycloepoxydon tepoxalin, or a combination thereafter.

23. The method as in claim 17, wherein the method of sterilization is Electronic beam (E-beam).

24. The method as in claim 17, wherein the chitosan in the mixture is crosslinked by E-beam radiation to increase viscosity of the mixture.
